(19) United States
(12) Patent Application Publication
(10) Pub. No.: US 2026/0306578 A1
(43) Pub. Date: Oct. 8, 2026

(54) Bio-Based Silica and Uses Thereof

(71) Applicant: SIL'INNOV SRL, Courcelles (BE)

(72) Inventor: Christiaan WILLAERT, Courcelles (BE)

(21) Appl. No.: 19/483,488

(22) PCT Filed: May 13, 2024
(86) PCT No.: PCT/EP2024/063103

§ 371 Date: Nov. 11, 2025

(30) Foreign Application Priority Data
May 12, 2023 (BE) 2023/5395

(51) Int. Cl. A61K 36/899

(52) U.S. Cl. CPC A61K 36/899

(57) ABSTRACT

The current invention relates to a composition comprising a biobased mesoporous silica with a specific surface area, calculated from nitrogen sorption isotherms at liquid nitrogen temperature (77K) by the Brunauer, Emmett and Teller (BET) theory, of at least 300 m2/g, wherein said mesoporous silica is impregnated with a compound. In addition, the invention relates to an oral dosage form, the use of said composition and a method for producing said composition.

### FIELD OF THE INVENTION

The present invention relates to a composition and oral dosage form comprising mesoporous silica derived from a biological source, more particularly from bamboo. In another aspect, the present invention also relates to a method for preparing said composition and oral dosage form.

### BACKGROUND

Bioavailability is a critical factor in determining the efficacy of many therapeutic agents and nutraceuticals. Poor bioavailability can limit the therapeutic or other effects of these compounds and often requires high doses or frequent administration, which can lead to unwanted side effects.

Mesoporous silica is a widely used material for improving the bioavailability of active ingredients due to its high surface area and pore volume. However, the use of chemically synthesized mesoporous silica has some drawbacks, such as the potential for toxicity and the need for costly and complex synthesis procedures. There is a need for an eco-friendly and more simple process for producing mesoporous silica.

Bamboo is a versatile plant with a long history of use in traditional medicine. Multiple studies have demonstrated that bamboo is a rich source of silica.

The present invention provides a method for enhancing the effectiveness of a wide range of biological active ingredients by formulating them with a bamboo silica extract. The extract is obtained by a simple and cost-effective process that preserves the bioactivity of the silica and other components. By incorporating the bamboo silica extract into formulations containing biological active ingredients, the present invention provides a simple and effective way to improve the bioavailability of these compounds, while avoiding the potential drawbacks associated with chemically synthesized mesoporous silica.

### SUMMARY OF THE INVENTION

The present invention and embodiments thereof serve to provide a solution to one or more of above-mentioned disadvantages. To this end, the present invention relates to a composition according to the first aspect. In a second aspect, the present invention relates to an oral dosage form comprising a composition according to the first and/or sixth aspect. In a third aspect, the invention relates to a composition in accordance with the first and/or sixth aspect, for use as a medicine, more particularly for use in the treatment and/or prevention of cancer, inflammatory diseases, neurological disorders, neurodegenerative diseases, cardiovascular diseases, metabolic disorders, obesity or cutaneous diseases.

In a fourth aspect, the invention relates to the use of any of the compositions herein provided to increase the bioavailability of a compound. In the first aspect, it is detailed how this can be achieved for a wide range of compounds. In the sixth aspect, it is detailed how this can be achieved for silicium itself. It is evident these teachings can readily be combined to achieve high silicium and additional target compound solubilities.

In a fifth aspect, the invention relates to a method of producing the compositions according to the invention.

In a sixth aspect, the invention relates to a composition comprising soluble silicium in accordance with claim 1. In particular, said composition comprises bio-based silicium, preferably derived from bamboo, wherein the composition was modified to improve the solubility and bioavailability of the silicium therein. In a seventh aspect, the invention relates to methods for producing compositions according to the sixth aspect in accordance with claims 8 and 15.

### BRIEF DESCRIPTION OF THE FIGURES

FIG. 1 shows the physisorption curves of the bamboo silica extract at the temperature of the liquid nitrogen. The black curve corresponds to the adsorption phase of the gas on the surface of the solid while the grey curve corresponds to the desorption phase. The hysteresis observed between the two curves is generally attributed to adsorption in mesoporous materials with capillary condensation.

FIG. 2 illustrates the pore sizes distribution of the bamboo silica extract.

FIG. 3 shows the particle size distribution in volume of the bamboo silica extract as determined with the Beckman Coulter method in water at room temperature.

FIG. 4 shows the X-ray reflections of the phloretin extracted from the dried Malus pumila root bark.

FIG. 5 shows the comparison of the X-ray reflections of the phloretin solid dispersion 303DS02 (grey) and of the physical blend among phloretin pure crystalline material and bamboo silica extract (black).

FIG. 6 illustrates the comparison of the phloretin kinetics release of the solid dispersion 303DS02 (dark grey) and the phloretin pure crystalline material (light grey).

FIG. 7 shows the comparison of the phloretin kinetics release of the solid dispersion 303DS02 with bamboo silica extract as carrier (dark grey) and the solid dispersion 302DS01 with a synthetic amorphous mesoporous silica (OSI100) as carrier (light grey).

FIG. 8 shows the X-ray reflections of trans-resveratrol (>98%) extracted from the root of the Polygonum cuspidatum.

FIG. 9 shows the comparison of the X-ray reflections of 303DS06 (dark grey) and of the physical blend among trans-resveratrol pure crystalline material and bamboo silica extract (light grey).

FIG. 10 shows the comparison of the trans-resveratrol kinetics release of 303DS06 (light grey) and the trans-resveratrol pure crystalline materials (dark grey).

FIG. 11 shows the comparison of the trans-resveratrol kinetics release of the solid dispersion 303DS06 with bamboo silica extract as carrier (light grey) and the solid dispersion 303DS05 with a synthetic amorphous mesoporous silica (OSI100) as carrier (dark grey).

FIG. 12 shows the amount of Si dissolved after 6 (left) and 8 (right) hours in FaSSIF at 37° C. from compositions A (black), B (dark grey), C, D and E (light grey) (from left to right).

FIG. 13 shows the dissolution kinetics of composition F in ultrapure water at a temperature ranging from 23 to 60° C.

FIG. 14 shows the evolution of the Si content for composition F in ultrapure water in function of the temperature.

FIG. 15 displays the amount of silicium (mg) dissolved in FaSSIF at 37° C. after 8 hours in function of the ball milling time.

FIG. 16 displays the amount of silicium (mg) dissolved in FaSSIF at 37° C. after 8 hours in function of the BPR ratio at the aforementioned milling conditions.

FIG. 17 displays the median particle size in function of the BPR ratio.

FIG. 18 displays the amount of silicium (mg) dissolved in FaSSiF at 37° C. after 8 hours, in function of the milling time (h).

### DETAILED DESCRIPTION OF THE INVENTION

The present invention concerns a composition, more particularly a man-made composition, comprising a bamboo silica extract and a biologically active compound.

The present invention further concerns a composition, comprising bamboo silica extract, wherein said composition has an increased silicium dissolution rate.

The inventors of current application demonstrated that the bio-availability of the biologically active compound with a bamboo silica extract as carrier is surprisingly comparable to that of a composition comprising the biologically active compound with a synthetic mesoporous silica as carrier. Interestingly, the use of bamboo silica extract enables an eco-friendly and more simple procedure of obtaining compositions comprising mesoporous silica that stimulate the bio-availability of biologically active compounds.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

Based on IUPAC recommendations for characterization of porous solids, porous materials are classified based on their pore size diameter. Pore diameter is defined as the average distance between pore walls. This classification does not contain any information of pore morphology e.g. geometry, orientation, interconnectivity of pores, etc. Porous materials classification, according to IUPAC, is as follows: microporous (<2 nm), mesoporous (2-50 nm), and macroporous (>50 nm) (Loni 2014 Springer International Publishing Switzerland).

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “w %”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

In a first aspect, the invention relates to a composition. More particularly a man-made composition or non-natural composition. The composition comprises a bamboo silica extract and a compound, more particularly a biologically active compound. In another embodiment, the composition is provided wherein the ratio of the total amount of the compound to the bamboo silica extract by weight is between 30/70 and 80/20, or between 10/90 and 50/50, preferably between 20/80 and 40/60 or between 25/75 and 35/65, even more preferably around 30/70. In a further or other embodiment, the total amount of the (biologically active) compound naturally present in the bamboo silica extract is less than 10 w %, less than 5 w %, less than 2 w %, less than 1 w %, less than 0.5 w % or less than 0.1 w % of the total weight of the bamboo silica extract. Hence, throughout current application, the composition is always a chimeric or unnatural composition comprising a bamboo silica extract and a (biologically active) compound, wherein the (biologically active) compound is completely or partially supplemented to the bamboo silica extract.

In a preferred embodiment, the biological active compound is not naturally present in the bamboo silica extract, more particularly the compound is derived from a source different than bamboo.

In a preferred embodiment of the invention, said bamboo silica extract comprises mesoporous silica in an amount of at least 60 wt %, more particularly comprises at least 70 wt %, at least 71 wt %, at least 72 wt %, at least 73 wt %, at least 74 wt % or at least 75 wt % mesoporous silica, relative to the total weight of the bamboo silica extract.

In another embodiment, the application also provides a composition comprising a biobased or bio-derived mesoporous silica and a compound. Bio-based or bio-derived mesoporous silica as used herein refers to mesoporous silica obtained from a biological source, for example an extract from a plant or algae, more particularly obtained from bamboo, even more preferably obtained from bamboo stem, bamboo leaves or young bamboo shoots. Most preferably, the silica is extracted from bamboo leaves or young shoots.

In another or a further embodiment, the composition of the invention comprises mesoporous silica, derived from a bamboo silica extract, in an amount of at least 30 wt %, at least 35 wt %, at least 40 wt %, at least 45 wt %, at least 50 wt % or at least 55 wt %, relative to the total weight of said composition, preferably around 49 wt %, around 50 wt %, around 51 wt %, around 52 wt % or around 53 wt %, more preferably in an amount of between 48 and 54 wt %, or between 50 and 53 wt % relative to the total weight of the composition.

The biobased mesoporous silica or mesoporous silica present in the bamboo silica extract of the invention preferably has a specific surface area, calculated from nitrogen sorption isotherms at liquid nitrogen temperature (77K) by the Brunauer, Emmett and Teller (BET) theory, of at least 250 m²/g, preferably at least 300 m²/g, more preferably at least 325 m²/g, at least 350 m²/g, at least 375 m²/g, at least 400 m²/g, at least 425 m²/g, at least 450 m²/g, at least 475 m²/g, at least 500 m²/g, at least 525 m²/g, at least 550 m²/g, at least 575 m²/g, at least 600 m²/g, at least 625 m²/g, at least 650 m²/g, at least 675 m²/g, at least 700 m²/g, at least 725 m²/g, at least 750 m²/g, at least 775 m²/g, at least 800 m²/g.

Even more preferably the specific surface area is between 300 m²/g and 900 m²/g, or between 300 m²/g and 800 m²/g, or between 300 m²/g and 700 m²/g, or between 300 m²/g and 650 m²/g, or between 300 m²/g and 600 m²/g, or between 300 m²/g and 500 m²/g, or between 300 m²/g and 450 m²/g, or between 350 m²/g and 450 m²/g, or between 375 m²/g and 425 m²/g. The inventors have surprisingly found that this specific surface area results in a similar release of a (biologically active) compound when dissolved in water compared to that of a synthetic amorphous mesoporous silica (purity>99%; OSI100) with a significantly higher specific surface area of 680 m²/g and the same content of the (biologically active) compound.

In a further embodiment, the biobased mesoporous silica or mesoporous silica present in the bamboo silica extract of the invention has mesopores having an average pore size of at least 3 nm. The pore size was calculated from nitrogen sorption isotherms at liquid nitrogen temperature (77K) according to the method of Barrett, Joyner and Halenda (BJH). The size of the mesopores of bamboo mesoporous silica, established from the isotherm of nitrogen sorption at 77K, is 4, 5, 6, 7, 8, 9, 10, 11, 12, 13, 14, 15, 16, 17, 18, 19, 20, 21, 22, 23, 24, 25, 26, 27, 28, 29 or 30 nm or any value between the above values.

In a particular embodiment, said average pore size calculated from nitrogen sorption isotherms at liquid nitrogen temperature (77K) according to the method of Barrett, Joyner and Halenda (BJH), of between 3 and 30 nm, preferably of between 3.5 and nm, preferably of between 4 and 15, preferably of between 4.5 and 12 nm or preferably of between 5 and 11 nm. In a particular embodiment, the mesopores of the mesoporous silica present in the bamboo silica extract of the invention have a distribution centered on round 7 nm, around 7.5 nm, around 8 nm, around 8.5 nm, around 9 nm, around 9.5 nm or around 10 nm. In a preferred embodiment, said distribution is centered around 8.5 nm.

In a further embodiment, the biobased mesoporous silica or mesoporous silica present in the bamboo silica extract of the invention has an average pore volume or porous volume of at least 0.1 cm³/g. In a preferred embodiment, the pore volume of the silica was calculated from nitrogen sorption isotherms at liquid nitrogen temperature (77K) by the method of Barrett, Joyner and Halenda (BJH). Said pore volume is at least 0.1 cm³/g, 0.2 cm³/g, 0.4 cm³/g, 0.5 cm³/g, at least 0.6 m³/g or at least 0.7 m³/g or any value included between the above values. In a preferred embodiment, said pore volume is around 0.7 m³/g.

In a further embodiment, the bamboo silica extract of the invention comprises particles that fall apart in two size distributions, more particularly in a size distribution centered at around 7 μm and in a size distribution centered at around 120 μm, determined by a particle size analyzer with the Beckman Coulter method in water at room temperature.

In a further embodiment, the bamboo silica extract of the invention comprises particles with a median particle size (d50), as determined by Beckman Coulter method in water at room temperature of less than 150 μm, of less than 125 μm, of less than 100 μm, of less than 75 μm, of less than 50 μm, of less than 25 μm or of less than 15 μm. In a further embodiment, the bamboo silica extract of the invention comprises particles with a median particle size (d50), as determined by Beckman Coulter method in water at room temperature of between 15 and 150 μm, more particularly of between 15 and 120 μm, even more particularly of around 100 μm.

In a further embodiment, the bamboo silica extract of the invention comprises particles characterized by a d10 of between 5 and 30 μm, as determined by Beckman Coulter method in water at room temperature, or of between 10 and 30 μm, even more particularly of around 20 μm.

In a further embodiment, the bamboo silica extract of the invention comprises particles characterized by a d25 of between 20 and 100 μm, as determined by Beckman Coulter method in water at room temperature, or of between 50 and 80 μm, even more particularly of around 70 μm.

In a further embodiment, the bamboo silica extract of the invention comprises particles characterized by a d75 of between 100 and 200 μm, as determined by Beckman Coulter method in water at room temperature, or of between 125 and 175 μm, even more particularly of around 145 μm.

In a further embodiment, the bamboo silica extract of the invention comprises particles characterized by a d90 of between 100 and 200 μm, as determined by Beckman Coulter method in water at room temperature, or of between 150 and 185 μm, even more particularly of around 180 μm.

In a further embodiment, the bamboo silica extract particle size distribution is characterized by a d10 of lower than 30 μm; d25 lower than 100 μm; d50 lower than 125 μm; d75 lower than 150 μm and d90 lower than 200 μm, wherein the particle sizes are determined by Beckman Coulter method in water at room temperature.

Lower particle sizes lead to significantly higher dissolution rates. Without being bound by theory, it is assumed that this effect may be caused by the significant increase in surface area, a decrease in surface tension, an associated decrease in crystal formation and lattice energy.

In the examples of current application it is demonstrated that ball-milling of the silica extract significantly decreases the particle size and that those decreased particle sizes for example lead to increased bioavailability of silicon or increased bioavailability of the compound that is physically blended with the bamboo silica extract. Therefore, the composition as described herein is provided wherein the bamboo silica extract comprises particles with a median particle size (d50), as determined by Beckman Coulter method in water at room temperature of between and 25 μm, more particularly of between 10 and 20 μm, even more particularly of around 15 μm.

In a further embodiment, the particles are characterized by a d10 of between 1 and 5 μm, as determined by Beckman Coulter method in water at room temperature, or of between 2 and 4 μm, even more particularly of around 3 μm. In a further embodiment, the particles are characterized by a d25 of between 2 and 10 μm, as determined by Beckman Coulter method in water at room temperature, or of between 4 and 8 μm, even more particularly of around 6 μm. In a further embodiment, the particles are characterized by a d75 of between 15 and 50 μm, as 10 determined by Beckman Coulter method in water at room temperature, or of between 20 and 40 μm, even more particularly of around 30 μm. In a further embodiment, the particles are characterized by a d90 of between 60 and 100 μm, as determined by Beckman Coulter method in water at room temperature, or of between 70 and 90 μm, even more particularly of around 80 μm. In a further embodiment, the particles are characterized by a d10 of at most 5 μm, a d25 of at most 10 μm, a d50 of at most 20 μm, a d75 of at most 40 μm and/or a d90 of at most 100 μm, wherein the particle sizes are determined by Beckman Coulter method in water at room temperature.

The (biologically active) compound may be sourced and purified from nature or may be synthesized. In a particular embodiment, the (biologically active) compound is a polyphenol. In another embodiment, the (biologically active) compound is an antioxidant.

An “antioxidant” as used herein refers to a molecule that has the ability to neutralize free radicals in the body. Free radicals are molecules with an unpaired electron, which makes them highly reactive and able to cause damage to cells and DNA in the body. Antioxidants work by donating an electron to a free radical, which stabilizes the radical and prevents it from causing damage to other molecules. This process of donating an electron to neutralize a free radical is called oxidation, and antioxidants are so named because they prevent or slow down the oxidation of other molecules.

In another embodiment, the (biologically active) compound is selected from the list consisting of antioxidants, polyphenols, minerals, vitamins, pro-vitamins, enzymes, probiotic bacteria, prebiotics, nutraceuticals, amino acids, algae extracts and plant extracts. Non-limiting examples of vitamins are vitamin C, vitamin E. A non-limiting example of a pro-vitamin is beta-carotene, which is converted into a vitamin (in this case into vitamin A) once taken up by the body.

Non-limiting examples of polyphenols are phloretin, phloretin derivative such as a glycosylated phloretin, resveratrol, trans-resveratrol, ellagic acid, curcumin, hydroxytyrosol, isoflavones, or flavonoids such as quercetin, catechin, epicatechin or anthocyanin.

“Phloretin” as used herein refers to the dihydrochalcone with CAS number 60-82-2 and IUPAC name 3-(4-hydroxyphenyl)-1-(2,4,6-trihydroxyphenyl)-propan-1one.

“Resveratrol” is a natural compound found in certain plants, particularly in the skin of red grapes, blueberries, cranberries, and peanuts. It belongs to a class of compounds called polyphenols, which are known for their antioxidant properties. Studies have suggested that resveratrol may have a number of potential health benefits. Resveratrol has been shown to reduce inflammation in the body, which is believed to be a contributing factor to many chronic diseases, including cancer, diabetes, and heart disease. Resveratrol has also been shown to have a positive effect on heart health by reducing the risk of heart disease, lowering blood pressure, and improving cholesterol levels. Also anti-aging effects are attributed to resveratrol as it has been shown to activate genes that are linked to longevity and may help to slow the aging process. Some studies have suggested that resveratrol may have anti-cancer properties by inhibiting the growth of cancer cells and promoting the death of cancer cells.

“Trans-resveratrol” is a specific form of resveratrol that is found in nature and is considered the most biologically active form of the compound. Trans-resveratrol is more stable and more easily absorbed than cis-resveratrol, which means it is less likely to break down or degrade over time and will be more utilized by the body when taken as a supplement.

In a specific embodiment, the (biologically active) compound can be a natural extract or obtained from natural extracts. In a specific embodiment, the (biologically active) compound is phloretin derived from apple trees (Malus, preferably Malus pumila), strawberry (Fragaria) or apricot trees (Prunus, preferably Prunus mandshurica). In a further preferred embodiment, the phloretin is produced from the leaves, bark or root of a fruit plant, preferably the aforementioned fruit plants. Most preferably, root of a fruit plant, in particular the root of Malus pumila. The root of Malus pumila contains a relatively high concentration of phloretin. More importantly, it can effectively be purified to arrive at a highly pure phloretin extract. Advantageously, naturally sourced phloretin is ecologically beneficial. Naturally sourced phloretin extracts generally comprise a mixture of phloretin and glycosylated phloretin. Glycosylated phloretin can be converted to phloretin by hydrolysis, such as enzymatic hydrolysis or acidic hydrolysis. Enzymatic hydrolysis provides ecological benefits. Acidic hydrolysis may provide for higher conversion and purity of product.

The phloretin derivative is preferably a glycosylated phloretin. In a further preferred embodiment, the glycosylated phloretin is chosen from the list consisting of: phlorizin, phloretin 3′,5′-di-C-glucoside and naringin dihydrochalcone. In an even further preferred embodiment, the glycosylated phloretin is phlorizin.

A “glycoside” is a molecule in which a sugar is bound to another functional group via a glycosidic bond. In formal terms, a glycoside is any molecule in which a sugar group is bonded through its anomeric carbon to another group via a glycosidic bond. Glycosides can be linked by an O- (an O-glycoside), N- (a glycosylamine), S- (a thioglycoside), or C- (a C-glycoside) glycosidic bond. According to the IUPAC, the name “C-glycoside” is a misnomer; the preferred term is “C-glycosyl compound”. By preference, the sugar is bonded to a non-sugar for the molecule to qualify as a glycoside, thus excluding polysaccharides. The sugar group is then known as the glycone and the non-sugar group as the aglycone or genin part of the glycoside. The glycone can consist of a single sugar group (monosaccharide) or several sugar groups (disaccharide or oligosaccharide).

“Glycosylated phloretin” and “glycoside of phloretin” as used herein are synonyms and refer to a molecule in which one or more sugars are bound to one or more functional groups of a phloretin molecule via a glycosidic bond. The glycosides can be linked by an O- (an O-glycoside), or C- (a C-glycoside) glycosidic bond. The sugar is preferably a monosaccharide or a disaccharide. Preferably, the one or more sugar is glucose.

“Phlorizin” as used herein refers to a glucoside of phloretin with CAS number 60-81-1 and IUPAC name 1-(2,4-Dihydroxy-6-{[(2S,3R,4S,5S,6R)-3,4,5-trihydroxy-6-(hydroxymethyl) oxan-2-yl]oxy}phenyl)-3-(4-hydroxyphenyl) propan-1-one.

In another specific embodiment, the (biologically active) compound can be a natural extract, more particularly a plant or algae extract, even more particularly a marine algae extract, even more particularly a marine algae extract rich in calcium and magnesium. A non-limiting example of a marine algae is a calcareous marine algae of the Lithothamnion genus.

In an even more specific embodiment, the algae extract, more particular marine algae extract comprises at least 15%, at least 20%, at least 25%, at least 30% calcium. In another specific embodiment, the algae extract, more particular marine algae extract comprises at least 1%, at least 1.5%, at least 2% magnesium. In an even more specific embodiment, the algae extract, more particular marine algae extract comprises at least 25% calcium and 1.5% magnesium or at least 30% calcium and 2% magnesium.

The inventors have surprisingly found that the addition of the algae extract, drastically increases the dissolution rate of the mesoporous silica present in the bamboo silica extract which allows the composition to provide bioavailable silicon. In a further embodiment, the composition provided herein comprises an algae extract or the mineral derived thereof and the bamboo silica extract as disclosed herein, wherein the algae extract (or the mineral derived thereof) and the bamboo extract are present in a 40:60 to 80:20 ratio.

In a further preferred embodiment, the mesoporous silica present in the bamboo silica extract, preferably in combination with minerals or algae extract, are added to water and subsequently heated to an elevated temperature. Preferably, the elevated temperature is between 5° and 99° C., more preferably between 6° and 99° C., more preferably between 65 and 95° C., more preferably between 7° and 90° C. The elevated temperature improves dissolution kinetics as well as solubility equilibrium to favor bioavailable silicon species over silica compared to room temperature. Furthermore, the inventors surprisingly found the addition of minerals, particularly Calcium and Magnesium ions (Ca²⁺ and Mg²⁺) limit the formation of silica gel upon cooling. Consequently, an aqueous solution with higher bioavailable silicon content is obtained. In a preferred embodiment, a supersaturated aqueous solution of silicon in the presence of minerals is obtained. As additional benefit, the aqueous solution with high bioavailable silicon content can be produced in a bio-based manner. Preventing polymerization of (bioavailable) silicon compounds is typically achieved through the use of synthetic organosilicon compounds, often in combination with high amounts of stabilizers, such as alkylated silanol compounds. The carbon-silicon bonds are more easily cleaved and absorbed than silica networks. However, this implies intake of synthetic organosilicon compounds which are often new and relatively untested.

The (biologically active) compound can be impregnated or loaded onto and/or into the mesoporous silica present in the bamboo silica extract in various ways. For example, the one or more compounds may be deposited onto the surface of the silica particles, incorporated into the pores of porous silica, incorporated into the pores formed by the agglomeration or consolidation of silica particles (particularly nanoparticles) or bound or otherwise associated with the surface of the bamboo silica. Suitable methods for loading the compound with the bamboo silica include the techniques of pan coating, fluidised bed, spray drying, spray chilling, enrobing, dusting/breading, coextrusion.

Higher levels of loading, for example, at least about 15 wt. % of the loaded compound based on the loaded weight of the bamboo silica extract may be achieved by performing the impregnation at an elevated temperature. For example, loading may be carried out at a temperature which is at or above the melting point of the compound to be loaded. Quantification of gross loading may conveniently be achieved by a number of known analytical methods, including gravimetric, EDX (energy-dispersive analysis by x-rays), Fourier transform infra-red (FTIR), Raman spectroscopy, UV spectrophotometry, titrimetric analysis, HPLC or mass spectrometry. If required, quantification of the uniformity of loading may be achieved by techniques that are capable of spatial resolution such as cross-sectional EDX, Auger depth profiling, micro-Raman and micro-FTIR.

The loading levels can be determined by dividing the weight of the compound taken up during loading (equivalent to the mass of the ingredient taken up divided by its density) by the void volume of the porous bamboo silica prior to loading multiplied by one hundred.

In a preferred embodiment of the invention, the composition is provided wherein at least 60 wt. % of the one or more (biologically active) compounds is dissolved in water after 2 hours at 37° C. This means that when the composition of the current invention is brought in water at 37° C., at least 60 wt. % of the one or more biologically active compounds is dissolved in water after 2 hours. In a further preferred embodiment, at least 65 wt. %, more preferably at least 70 wt. %, more preferably at least 75 wt. %, more preferably at least 80 wt. %, more preferably at least 85 wt. %, more preferably at least 90 wt. %, more preferably at least 95 wt. % of said one or more biologically active compounds is dissolved in water after 2 hours at 37° C. This high amount of the dissolved (biologically active) compound or compounds results in a higher bioavailability.

In another preferred embodiment of the invention, the (biologically active) compound, which is impregnated in or on the mesoporous silica has an XRD crystallinity of at most 80%, preferably at most 70%, more preferably at most 60%, more preferably at most 50%, more preferably at most 40%, more preferably at most 35%, more preferably at most 30%, more preferably at most 25%, more preferably at most 20%, more preferably at most 15%, more preferably at most 10%, more preferably at most 8%, more preferably at most 7%, more preferably at most 6%, most preferably at most 5%.

The XRD crystallinity degree of the samples can be determined by the method of X-ray diffraction as described below. The inventors found that lower XRD crystallinity was associated with a significant increase in the dissolution rate.

In another preferred embodiment, the compound present in the composition is a salt or the composition further comprises a salt. Said salt consists of a cation and an anion, wherein said salt is chosen from the list of alkali metal salts, alkaline earth metal salts or a mixture thereof. More preferably, the salt is chosen from the list of magnesium salts, calcium salts, potassium salts, sodium salts or a mixture thereof, more preferably magnesium salts or calcium salts, most preferably calcium salts. Preferably, said salt is an organic salt, preferably said salt is an acetate, citrate, tartrate, formate, benzoate, gluconate, sorbate or mixture thereof. Most preferably, said salt is calcium acetate. Preferably, said salt is edible, more preferably said salt is pharmaceutically acceptable. Preferably, the ratio by weight of the cation of said salt to the bamboo silica extract lies between 1:100 and 200:100, preferably the ratio by weight of the cation of said salt to the bamboo silica extract lies between 10:100 and 200:100, more preferably between 50:100 and 150:100, even more preferably between 40:60 and 80:20. The inventors have surprisingly found that the addition of these salts, more particularly salts present in an algae extract, presumably due to stabilizing effects of silicic acids such as orthosilicic acid associated with the dissolved cations once in aqueous environment, drastically increases the dissolution rate of the mesoporous silica present in the bamboo silica extract. This advantageously allows the composition to provide bioavailable compounds as well as bioavailable silicium. Furthermore, the dissolution of the mesoporous silica from the bamboo silica extract aids in the dissolution of the biologically active compound particles.

In a further embodiment, the bamboo silica extract present in the compositions herein provided, is an extract of young leaves of bamboo. “Young” as used herein, refers to leaves between 1 and 10 days old, or between 5 and 14 days old or between 2 and 20 days old. In another particular embodiment, the leaves are less than 100 days old, less than 80 days old, less than 60 days old, less than 40 days old, less than 21 days old or less than 15 days old.

In another further embodiment, the bamboo silica extract is a silica extract from leaves of Bambusa arundinaceae. A non-limiting example of a process of obtaining such extract comprises a step of harvesting the young bamboo leaves, a step of washing and drying the bamboo leaves, a step of grinding the dried bamboo leaves to a powder and optionally a step of treating the bamboo powder with a base to remove lignin and hemicellulose.

In a second aspect, the present invention provides an oral drug dosage form comprising any of the compositions according to the first aspect or described herein.

In a preferred embodiment, said oral dosage form is a solid oral dosage form such as a tablet or a powder, and more preferably said solid oral dosage form is a tablet.

For reasons of ease in dissolution behavior, said tablet is preferably not provided with an additional coating.

The inventors have found that the composition according to the first aspect is especially advantageous as an oral dosage form because the inventors have found that the composition according to the first aspect improves the bio-availability and thus the uptake of the one or more (biologically active) compounds present in the composition. In particular, these effects are pronounced within the gastro-intestinal tract.

In a preferred embodiment, the present invention provides an oral drug dosage form comprising said composition in an amount of at least 1 wt. %, relative to the total weight of said oral dosage form, preferably at least 10 wt. %, preferably at least 25 wt. %, preferably of at least 50 wt. %, preferably at least 80 wt. %.

A higher percentage of the composition according to the invention allows for higher concentration of silica and the to be delivered (biologically active) compound, in the gastro-intestinal tract of the subject, and thus allows for better uptake in the human or animal body.

As described above, the bamboo mesoporous silica increases the bio-availability of a compound that is loaded onto the silica particle, in particular when the particle size of the compound is reduced and the compound is mostly loaded in an amorphous state. Silica beneficially acts as a stabilizer for small, amorphous particles in this context. This is particularly useful when the compound is a medicine or has health promoting effects. Therefore in a third aspect, any of the compositions or any of the oral dosage forms herein disclosed, are provided for use as a medicament or for use to prevent or treat a disease. This is equivalent as saying that method of treating a subject are provided, wherein any of the compositions or any of the oral dosage forms herein disclosed are administered to a subject in need thereof.

Dihydrochalcone phloretin for example is able to accelerate remyelination. In particular, the phloretin is able to stimulate OPC maturation in in vitro OPCs cultures and improved (re) myelination in microglia-depleted ex vivo demyelinated brain slices. Furthermore, it has been shown here that phloretin is able to drive OPC differentiation via PPARy activation. Because of these effects on remyelination and oligodendrocyte differentiation and maturation, the present application provides in a further embodiment a composition according to the first aspect or an oral dosage form according to the second aspect for use in the treatment or prevention of a neurological disease, more particularly a demyelinating disease.

As used herein, the term “demyelinating disease”, is a disease condition in which the myelin sheath which surrounds neurons in nervous tissue is lost or damaged, leading to axonal degeneration and impaired signal transduction in the affected nerves. A demyelinating disease of the central nervous system is a disease in which the myelin sheaths of neurons in the central nervous system are lost or damaged. Examples of demyelinating diseases of the central nervous systems are multiple sclerosis, neuromyelitis optic (Devic's disease), inflammatory demyelinating diseases, central nervous system neuropathy, central pontine myelinolysis, myelopathy, leukoencephalopathy, or leukodystrophy.

A demyelinating disease of the peripheral nervous system is a disease condition in which the myelin sheaths of neurons in the peripheral nervous system are lost or damaged. Examples of demyelinating diseases of the peripheral nervous system are Guillain-Barre syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy, diabetic neuropathy or traumatic nerve injury. In another preferred embodiment of the third aspect, the invention relates to a composition according to the first aspect or an oral dosage form according to the second aspect to provide neuroprotective effects.

Other compounds can be of help in treating or preventing other diseases. Therefore, in a further embodiment of the third aspect, the composition herein disclosed is provided for use to treat or prevent inflammatory diseases, cancer, neurological diseases, neurodegenerative diseases, cardiovascular diseases, metabolic disorders, cutaneous diseases, hepatic diseases, bone diseases, estrogen deficiency, estrogen deficiency-induced osteoclastogenic resorption, obesity, cardiomyopathy, demyelinating diseases.

Non-limiting examples of neurodegenerative diseases are Alzheimer's disease, dementia, multiple sclerosis, Parkinson disease, ALS, Charcot-Marie-Tooth disease, Huntington disease, multiple system atrophy, traumatic nerve injury, diabetic neuropathy, chronic inflammatory polyradiculoneuropathy, Guillain Barre syndrome.

Non-limiting examples of cutaneous diseases are those related to the health and welfare of skin, for example skin ageing, hyperpigmentation, acne and skin cancer.

The subject may be a non-human animal or a human. Preferably, the subject is a human.

In a fourth aspect the use of biobased mesoporous silica is provided to increase the bioavailability of a compound. “Biobased” as used herein refers to “bio-derived” or “biologically derived” or “biologically based” and means that the mesoporous silica is obtained, extracted or derived from a biological source, such as a plant.

In a specific embodiment, the biobased mesoporous silica is silica obtained or extracted from bamboo, more particularly the biobased mesoporous silica is a bamboo silica extract, even more particularly a bamboo silica extract comprising at least 70% mesoporous silica.

Also provided is a method of improving the bioavailability of a compound such as a biologically active substance, comprising the steps of:
providing a biobased mesoporous silica;
loading the mesoporous silica from the bamboo silica extract with the compound;
administering the loaded mesoporous silica to a subject in need thereof;
wherein the bioavailability of the compound is increased compared to administering the biologically active compound alone.

Preferred embodiments of the compositions are in accordance with the first and sixth aspect as described herein; and produced through their respective methods. The inventors have found that by decreasing the particle size of the compound more particularly the biologically active compound during impregnation or physically blending in or on the mesoporous silica, the dissolution rate in water significantly increased. This higher dissolution rate results in a higher bio-availability, and especially a higher uptake of the biologically active compound in the gastro-internal tract.

In a particular embodiment, said loading is physically blending the mesoporous silica with the compound. In a further embodiment, physically blending comprises milling for example ball-milling or hammer-milling of the silica with the compound.

The mesoporous silica as mentioned in the above provided use and above provided method of improving the bioavailability of a compound, can be any mesoporous silica as described in the first aspect.

In another or a further preferred embodiment of the invention, the method comprises the steps of:
i. providing a biobased mesoporous silica, preferably with a specific surface area, calculated from nitrogen sorption isotherms at liquid nitrogen temperature (77K) by the Brunauer, Emmett and Teller (BET) theory, of at least 250 m²/g;
ii. providing a compound having a first XRD crystallinity, as determined using X-ray diffraction and Segal's formula;
iii. impregnating or physically blending said mesoporous silica with the compound, wherein said compound having a second XRD crystallinity, as determined using X-ray diffraction and Segal's formula, lower than said first XRD crystallinity.

In this embodiment of the invention, the ratio of said first XRD crystallinity to said second XRD crystallinity is at least 1.25, preferably at least factor 1.5, more preferably at least factor 2.

In a further embodiment of the invention, the first XRD crystallinity, as determined using X-ray diffraction and Segal's formula, of the compound is between 70 and 100%, preferably between 80 and 100%, more preferably between 90 and 100%.

In a further embodiment of the invention, the second XRD crystallinity, as determined using X-ray diffraction and Segal's formula, of the compound is at most 70%, more preferably at most 60%, even more preferably at most 50%, more preferably at most 40%.

The inventors have found that by decreasing the crystallinity of the compound during impregnation in or on the mesoporous silica from the bamboo silica extract the dissolution rate in water significantly increased. This higher dissolution rate results in a higher bio-availability, and especially a higher uptake of the compound in the gastro-internal tract. The more amorph the compound is, the better the dissolution in water.

### Sixth and Seventh Aspect

In a sixth aspect, the invention relates to a composition comprising a silica extract, wherein the composition has improved solubility of silicium (Si). Silicium and particularly silica is difficult to dissolve or solubilize and readily polymerizes and precipitates from water. Present disclosure aims to modify silica extracts derived from bamboo so the dissolution kinetics are favored. This allows for the production of bio-based silicium compositions which have a high content of bio-available, soluble silicium.

In an embodiment, the fifth aspect relates to a composition comprising a silica extract derived from bamboo, wherein said composition is characterized by a dissolution of silicium (Si) of at least 3 wt. %, more preferably at least 4 wt. %, more preferably at least 5 wt. %, more preferably at least 6 wt. %, more preferably at least 7 wt. %, more preferably at least 8 wt. %, more preferably at least 9 wt. %, more preferably at least 10 wt. %, more preferably at least 11 wt. %, more preferably at least 12 wt. %, more preferably at least 13 wt. %, more preferably at least 14 wt. %, more preferably at least 15 wt. % relative to the silicium in the composition, in FaSSIF after 8 hours at 37° C. as measured by ICP-OES.

In an embodiment, said composition comprises silica SiO2 in an amount of at least 30 wt. %, more preferably at least 40 wt. %, more preferably at least 50 wt. %, more preferably at least 60 wt. %, more preferably at least 70 wt. %, more preferably at least 80 wt. %, more preferably at least 90 wt. %, most preferably consists essentially of silica. Preferably, the silica comprised within said composition is bio-based, more preferably derived from bamboo, most preferably a bamboo silica extract. Advantageously, present disclosure teaches methods to strongly enhance the solubility of silica extracts from bamboo without the need for chemical conversion, solvents or (organochemical) silicium precursors and the ecological issues associated with these practices.

In an embodiment, said composition has a median particle size of at least 0.001 μm, more preferably at least 0.005 μm, more preferably at least 0.01 μm, more preferably at least 0.05 μm, more preferably at least 0.10 μm. In an embodiment, said composition has a median particle size of most 15.00 μm, more preferably at most 10.00 μm, more preferably at most 5.00 μm, more preferably at most 3.00 μm, more preferably at most 2.00 μm, more preferably at most 1.00 μm, more preferably at most 0.80 μm, more preferably at most 0.60 μm, more preferably at most 0.50 μm, more preferably at most 0.30 μm. In a preferred embodiment, said composition has a median particle size between 0.05 and 1.00 μm, more preferably 0.10 and 0.50 μm. The median particle size d50 is measured by the Beckman Coulter method in water at room temperature.

In an embodiment, said composition has a specific surface area calculated from nitrogen sorption isotherms at liquid nitrogen temperature (77K) by the Brunauer, Emmett and Teller (BET) theory, of at most 600 m²/g, more preferably at most 500 m²/g, more preferably at most 400 m²/g, more preferably at most 350 m²/g, more preferably at most 300 m²/g, more preferably at most 250 m²/g, more preferably at most 200 m²/g, more preferably at most 150 m²/g. In a preferred embodiment, the specific surface area of the composition is between 50 and 400 m²/g, more preferably between 100 and 300 m²/g. Compositions with a high specific surface area are typically easier to dissolve due to the large water-solids interface. However, obtaining bio-based, particularly bamboo based, silica with high specific surface area remains challenging. Typically chemical reformation is required. The inventors found that silica with low mesoporosity and low surface area can be solubilized. Furthermore, these methods further decrease the specific surface area, yet still result in great solubility of silicium therein.

In a preferred embodiment, the composition comprises silica in an amount of at least 50 wt. %, has a particle size between 0.1 and 1.0 μm and a specific surface area between 100 and 300 m²/g. Preferably the silica is entirely bio-based, more preferably extracted from bamboo. Preferably, the remainder of the composition is plant-based impurities from bamboo. Advantageously, these do not need to be removed in their entirety, reducing extraction purification requirements and drastically improving yields. Despite these limited purification and modification requirements, a composition with relatively high solubility of silicium into FaSSIF is obtained.

In the seventh aspect, present invention relates to a method for the production of a soluble silica composition. Particularly, the seventh aspect relates to a method to convert a silica extract, preferably a bio-based silica extract, into a composition with a much higher silicium solubility and silicium dissolution rate. In an embodiment, the method comprises the steps of:
providing a milling composition, said composition comprising a silica extract derived from bamboo in an amount of at least 30 wt. % relative to the composition; and
ball milling said milling composition, thereby obtaining a soluble silica composition.

In an embodiment, ball milling is performed at a ball-to-powder (BPR) ratio of at least 2.5, more preferably at least 3.0, more preferably at least 3.5, more preferably at least 4.0, more preferably at least 4.5, more preferably at least 5.5. In an embodiment, ball milling is performed at a ball-to-powder (BPR) ratio of at most 10.0, more preferably at most 8.0, more preferably at most 7.0, more preferably at most 6.5, more preferably at most 6.0, more preferably at most 5.5. In an embodiment, ball milling is performed at a ball-to-powder (BPR) ratio of between 3.5 and 7.0, more preferably between 4.5 and 6.0. The ball-to-powder ratio is determined as a weight-to-weight ratio of the milling balls relative to the milling composition in its entirety. Lower ball-to-powder ratios result in insufficiently low particle sizes and/or significantly longer milling times required. Higher ball-to-powder ratios are very energy inefficient.

Preferably, ball milling is not vibrational ball milling. Vibrational ball milling has high energy requirements with limited to no benefits. It is also not suitable for large batch production.

Ball milling is preferred over other milling mechanisms. Various other types of milling were investigated, such as hammer milling. These were found to be considerably less effective at solubilizing silicium from silica. While increases could be obtained, these required significantly more energy or the use of solvents and/or chemicals thus eliminating the ecological benefits of present methods.

In an embodiment, the ball milling time is at least 3.0 hours, more preferably at least 3.5 hours, more preferably at least 4.0 hours, more preferably at least 4.5. In an embodiment, the ball milling time is at most 8.0 hours, more preferably at most 7.0 hours, more preferably at most 6.0 hours, more preferably at most 6.5 hours, more preferably at most 6.0 hours, more preferably at most 5.5 hours, more preferably at most 5.0 hours. In an embodiment, the milling time is between 3.0 and 8.0 hours, more preferably between 3.5 and 5.0 hours. Most preferably, these ball milling times are utilized in combination with the BPR ratios previously described. The preferred milling times and BPR ratios are optimized to maximize dissolution rate in function of energy requirements. Dissolution rate can be further increased, but this comes at an increasingly large energy cost. Furthermore, at a certain point dissolution rate plateaus limiting further gains. In addition, further increasing the BPR ratio may result in limited stability. The very small particles agglomerate towards larger particle sizes during storage and transport, minimizing the benefits of further increasing the BPR and/or milling time.

In a preferred embodiment, the ball milling RPM is at least 100, more preferably at least 200, more preferably at least 300, more preferably at least 400. In a preferred embodiment, the balling milling RPM is at most 1000, more preferably at most 800, more preferably at most 700, more preferably at most 600, more preferably at most 500. Most preferably, the ball milling RPM is between 300 and 600, more preferably between 400 and 500.

In an embodiment, the milling composition has a specific surface area of at most 650 m²/g, more preferably at most 600 m²/g, more preferably at most 550 m²/g, more preferably at most 500 m²/g, more preferably at most 450 m²/g, more preferably at most 400 m²/g, more preferably at most 350 m²/g, more preferably at most 300 m²/g, more preferably at most 250 m²/g. In an embodiment, the milling composition has a specific surface area of at least 1 m²/g, more preferably at least m²/g, more preferably at least 25 m²/g, more preferably at least 50 m²/g, more preferably at least 100 m²/g, more preferably at least 150 m²/g, more preferably at least 200 m²/g. Preferably and advantageously, when ball milling starting from compositions with a relatively lower specific surface area is preferred. Ball milling at the required intensity reduces the specific surface area. Production of high specific surface area has higher energy requirements and specific production requirements. By starting from compositions with a lower specific surface area, the energy requirements and process complexity is avoided or at least reduced; with limited impact on the solubility of the result. This is particularly desirable when solubilizing bio-based products.

In an embodiment, the milling composition has a median particle size d50 of at least 1.0 μm, more preferably at least 1.5 μm, more preferably at least 2.0 μm, more preferably at least 2.5 μm, more preferably at least 3.0 μm, more preferably at least 3.5 μm, more preferably at least 4.5 μm, more preferably at least 5.0 μm, more preferably at least 7.5 μm, more preferably at least 10.0 μm, more preferably at least 12.5 μm, more preferably at least 15.0 μm, more preferably at least 20.0 μm, more preferably at least 25.0 μm, more preferably at least 50.0 μm. The median particle size is determined by Beckman Coulter method in water at room temperature.

In a preferred embodiment, the method comprises the steps of:
providing a milling composition, said composition comprising a silica extract derived from bamboo in an amount of at least 30 wt. % relative to the composition; wherein said milling composition has a median particle size d50 of at least 2.5 μm and a specific surface area lower than 300 g/m²; and
ball milling said milling composition at a BPR ratio by weight between 3.0 and 8.0, for a duration between 3.0 and 8.0 hours, thereby obtaining a soluble silica composition.

In a particular preferred embodiment, the method comprises the steps of:
providing an insoluble composition, said composition comprising a silica extract derived from bamboo in an amount of at least 30 wt. % relative to the composition; and
measuring the specific surface area calculated from nitrogen sorption isotherms at liquid nitrogen temperature (77K) by the Brunauer, Emmett and Teller (BET) theory of said insoluble composition;
when the specific surface area of the insoluble composition is above 650 m²/g, a salt, preferably calcium salt, is added to the insoluble composition in an amount of at least 10 wt. % relative to the weight of the silica extract; thereby obtaining the soluble silica composition; and
when the specific surface area of the insoluble composition is below 650 m²/g, said insoluble composition is ball milled; thereby obtaining the soluble silica composition.

The inventors surprisingly found relatively inexpensive yet highly effective methods to solubilize both high and low specific surface area bio-based silica extracts. This allows solubilization of silica extracts in general, without being restricted to for example silica's with high specific surface area. Furthermore, the methods do not require solvents or fossil-based chemical reactants. Thus a bio-based and ecologically favorable silica composition can be obtained which has high silicium bioavailability. In particular, the inventors found that alkali salts, particularly bivalent salts, most preferably calcium salts significantly improve the dissolution of mesoporous silica with a high specific surface area. However, the effect of calcium salts on silica without a high specific surface area is very limited. Silica any specific surface area can be solubilized by ball milling until a dry dispersion with a relatively small particle size is obtained. This process reduces the specific surface area of the silica and is energy-intensive. In addition, producing the structures that result in the very high specific surface areas also requires high amounts of energy. Thus ball-milling is a more energy-efficient method for silica's with a lower specific surface area. It is particularly desirable because most bio-based silica extracts, not withstanding specific growth and extraction conditions as well as later modification, have a specific surface area too low for the addition of calcium salts to be sufficiently effective.

The preferred conditions for ball milling are as previously described.

In a preferred embodiment, the salt is an organic salt, preferably a magnesium or calcium salt, more preferably a calcium salt. Preferably, said salt is an acetate, citrate, tartrate, formate, benzoate, gluconate, sorbate or mixture thereof. Most preferably, said salt is calcium acetate. In another preferred embodiment, the salt is a plant or algae extract. More preferably, the salt is a marine algae extract rich in calcium cations (Ca²⁺). A non-limiting example of a marine algae is a calcareous marine algae of the Lithothamnion genus. Advantageously, this provides a bio-based source of calcium in combination with a bio-based source of silicium with high solubility.

The inventors have surprisingly found that the addition of the algae extract, drastically increases the dissolution rate of the mesoporous silica present in the bamboo silica extract which allows the composition to provide bioavailable silicon. In a further embodiment, the composition provided herein comprises an algae extract or the mineral derived thereof and the bamboo silica extract as disclosed herein, wherein the algae extract (or the mineral derived thereof) and the bamboo silica extract are present.

In a further preferred embodiment, the soluble composition comprises silica to calcium salt, preferably calcium acetate or calcium algae extract, in a ratio between 20:80 and 80:20, more preferably 40:60 to 80:20, more preferably 50:50 to 80:20, most preferably 60:40 to 80:20 of silica to algae by weight.

The invention is further described by the following non-limiting examples which further illustrate the invention, and are not intended to, nor should they be interpreted to, limit the scope of the invention.

### EXAMPLES

### Example 1. A Bamboo Extract Comprising Mesoporous Silica

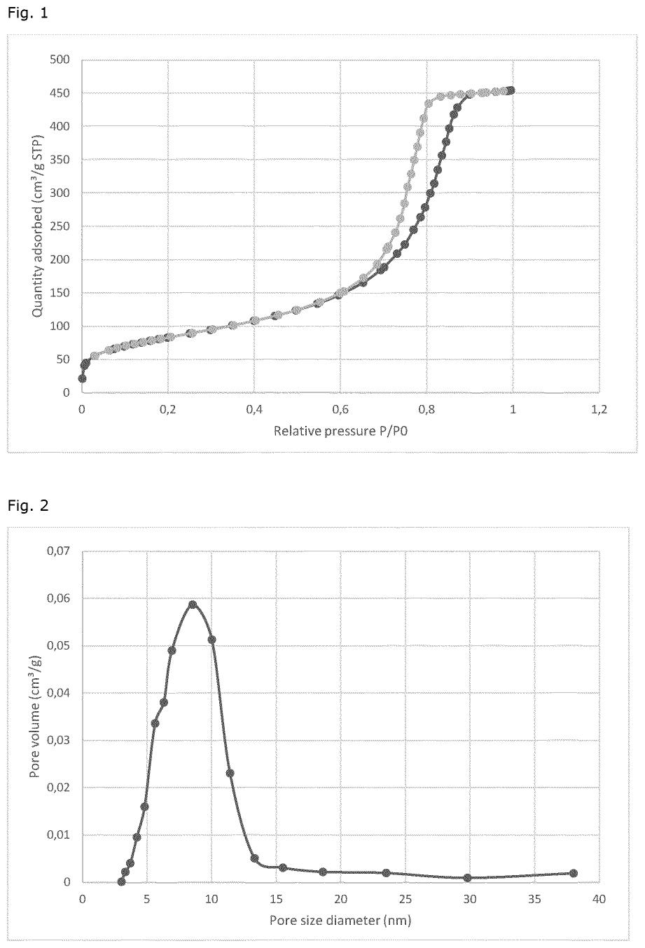

Young shoots from Bambusa arundinacea were harvested and used to make an extract. Surprisingly we found that the bamboo silica extract comprises a mesoporous material, more particularly mesoporous silica. The mesoporosity of the silica is shown by the nitrogen physisorption curves in FIG. 1. Interestingly, the bamboo silica extract comprises a very high amount of the mesoporous silica, more particularly 76.5% relative to the total weight of the bamboo silica extract.

Brunauer, Emmett and Teller (BET) specific surface area and pore size distribution measurements of the bamboo silica extract have been determined by an ASAP 2460 Surface Area and Porosity Analyzer from Micromeritics at the temperature of the nitrogen liquid (−198.8° C. or 77K). Nitrogen was used as gas analyzer.

The mesoporous bamboo silica extract is characterized with a high specific surface area of around 300 m²/g determined from the amount of nitrogen adsorbed. Given that the organic compounds present in the bamboo silica extract will negatively influence the specific surface of the material because their specific surface is likely to be very low, the porosity of the mesoporous silica present in the bamboo extract is higher than measured. Considering the silica content of 75% in the material, the actual specific surface area of the mesoporous silica present in the bamboo extract is around 400 m²/g.

The volume of the pores of the mesoporous silica by BJH desorption and adsorption method has been measured at 0.70 cm³/g.

The pore sizes distribution of the bamboo silica extract can be observed in FIG. 2, which shows dV/dW Pore Volume (cm³/g) in function pore size (nm). As shown in FIG. 2, the curve of the pore size distribution of the bamboo silica extract is centered on 8.5 nm.

The particle size distribution of the bamboo silica extract has been determined by a particle size analyzer with the Beckman Coulter method in water at room temperature. The particle size distribution in volume of the material with this method can be observed in FIG. 3. Two size distributions are observed, a minority one centered at 7 μm and a major one at approximately 120 μm.

The main parameters of the particle size distribution of this material are shown in Table 1. 10% of the particles have a size below 27.2 μm (d10), 25% of the particles have a size below 72 μm (d25), 50% of the particles have a size below 107 μm (d50), 75% of the particles have a size below 145 μm (d75) and 90% of the particles have a size below 181 μm (d90).

TABLE 1
Main parameters of the particle size distribution
in volume of the bamboo silica extract.

| d₁₀ (μm) | d₂₅ (μm) | d₅₀ (μm) | d₇₅ (μm) | d₉₀ (μm) |
|---|---|---|---|---|
| 27.2 | 72.0 | 107 | 145 | 181 |

### Example 2. A High Purity Apple Extract (>95% Phloretin)

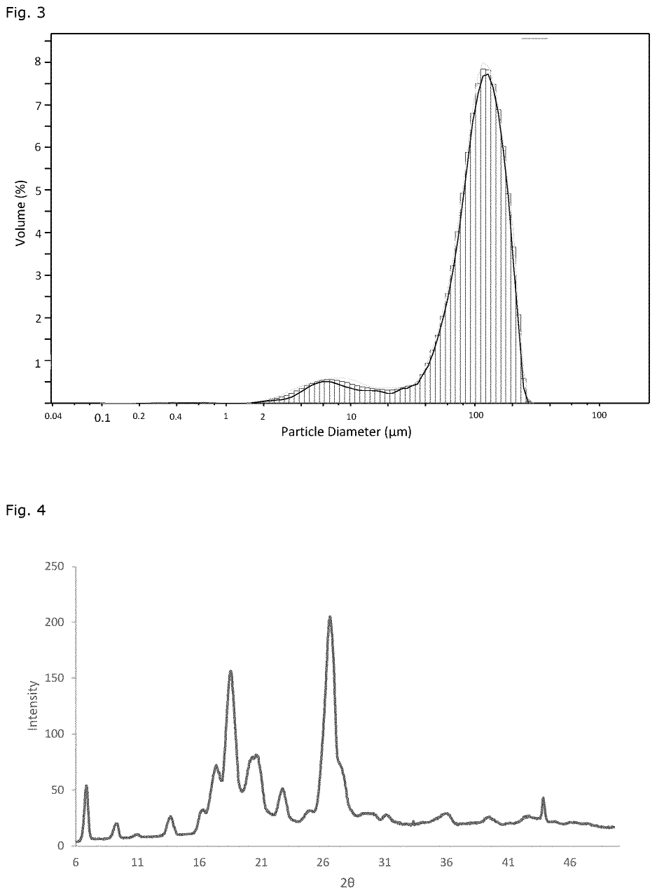

Phloretin is extracted from a dried Malus pumila root bark. The phloretin powder is a crystalline material as shown by the X-ray reflection in FIG. 4, which shows the intensity (I) of the X-ray reflections of the phloretin in function of 2θ, wherein 2θ is the angle between the incident ray and the reflected ray.

Characterization of the amorphous or crystalline state of phloretin was performed by X-ray diffraction (XRD) using a Bruker D2 Phaser diffractometer with Cu-Kα radiation. Sample were run over the range of 6° to 50° (2θ) with a step size of 0.04° at a speed of 2°/min.

### Example 3. Solid Dispersion of Phloretin and the Bamboo Extract

The bamboo extract, as described in example 1, and the phloretin, as described in example 2, were ball milled, in a ratio by weight of 70/30 to obtain a phloretin-based solid dispersion, with the following process conditions:
Ball Mill XQM-2 from TMAX (China).
Stainless steel balls (9 balls with diameter 1.5 cm; 22 balls with diameter 1.2 cm; 60 balls with diameter 1 cm and 100 balls with diameter 0.8 cm)
Speed of rotation: 450 rpm
Container volume: 500 ml
Time: 120 minutes
Single direction of rotation (no change of direction during grinding)

The phloretin-bamboo extract solid dispersion obtained by ball-milling (also referred to herein as composition 303DS02) has been characterized by X-ray diffraction and the reflections obtained have been compared to a physical blend of bamboo silica extract and phloretin pure crystalline material at the same phloretin content. The comparison of the reflection of both materials can be observed in FIG. 5, which shows the intensity (I) of the X-ray reflections of the composition in function of 2θ, wherein 2θ is the angle between the incident ray and the reflected ray.

Characterization of the amorphous or crystalline state of the composition was performed by X-ray diffraction (XRD) using a Bruker D2 Phaser diffractometer with Cu-Kα radiation. Sample were run over the range of 6° to 50° (2θ) with a step size of 0.04° at a speed of 2°/min.

The comparison of the X-ray reflections of the phloretin-bamboo extract solid dispersion and of the physical blend among phloretin pure crystalline material and bamboo silica extract shows that the ball-milling process amorphized the phloretin as the intensities of the characteristics phloretin X-ray reflections decrease after the ball milling process.

### Example 4. Comparison of the Phloretin Kinetic Release

The phloretin kinetic release of phloretin solid dispersion obtained by ball-milling with bamboo silica extract as carrier (example 3) has been compared to the phloretin kinetic release of the phloretin pure crystalline material (example 2) in the conditions of release described below:
Apparatus: European Pharmacopoeia dissolution apparatus type II (paddles)—AT Xtend (Sotax)
Dissolution medium: Ultrapure water
Volume medium: 900 ml
Speed of rotation: 75 rpm
Stirrer: paddles
Temperature medium: 37.0° C.±0.5° C.
Sampling volume: 5 ml filtered on PTFE membrane (0.45 μm)-syringe filter. Fresh medium (ultrapure water) is added on each flask where the sampling has been performed.
Sampling frequency: 0.25 h; 0.5 h; 1 h and 2 h
Phloretin dose per sample: 50 mg

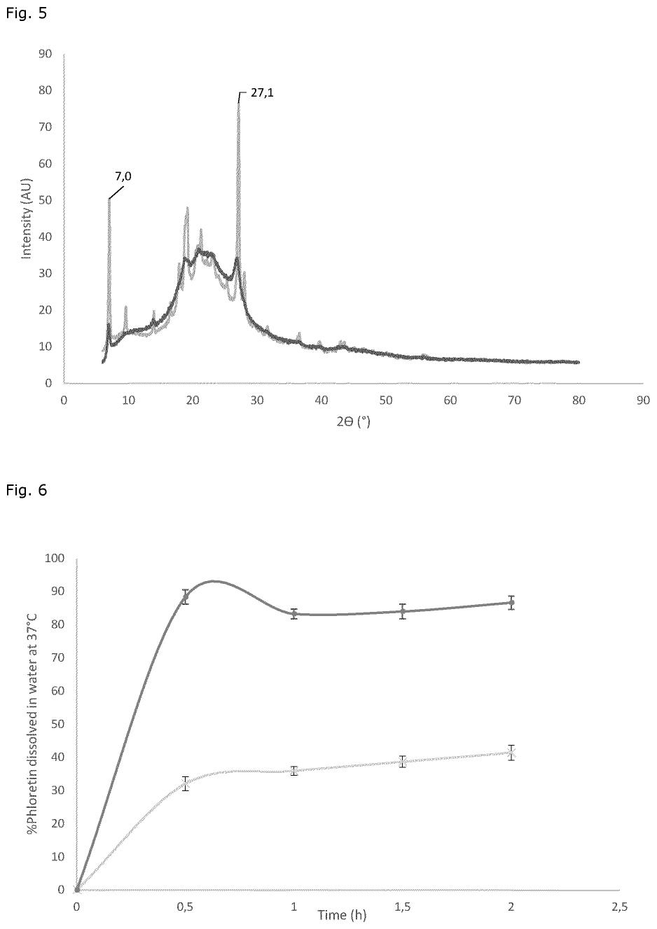

The comparison of the phloretin kinetics release of both products is shown in FIG. 6. The release trial shows that the amount of phloretin dissolved for the pure crystalline material reaches 40 wt. % of active dissolved after 2 hours at 37° C. in water. For the phloretin solid dispersion obtained with bamboo silica extract as carrier (303DS02) this amount reaches approximately 87 wt. %.

The amount of phloretin released is increased by a factor of 2.2 when it is in the form of a solid dispersion with the specific properties of the bamboo silica extract in terms of specific surface area, pore size distribution and particle size distribution as described in example 2.

Next, we compared the phloretin kinetic release of the solid dispersion with bamboo silica extract as carrier to that of a solid dispersion with a synthetic amorphous mesoporous silica as carrier. The synthetic silica (OSI100) had a high specific surface area of 680 m²/g and the same phloretin content (30 w %) than the phloretin solid dispersion with bamboo silica extract as carrier.

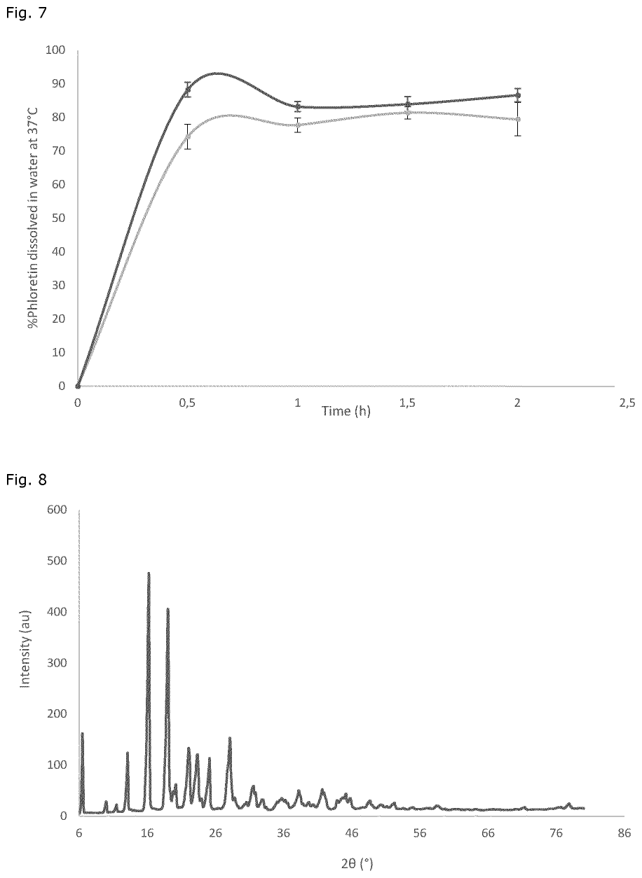

The comparison of the phloretin kinetics release of both products according to the release conditions described above, is shown in FIG. 7.

The kinetics release of both phloretin based solid dispersion materials (i.e. obtained with a synthetic amorphous mesoporous silica (OSI100) with a high specific surface area of 680 m²/g and the one obtained with the bamboo silica extract) are comparable, with the phloretin-bamboo extract dispersion releasing more phloretin during the first hour. From 1 hour onwards and considering the standard deviation of the trials the phloretin release kinetics are the same for both materials and after 2 hours the phloretin amount released is between 80 to 85% in both cases.

This is a surprising result since in general a higher specific surface area of a mesoporous material is directly correlated to a higher release of a compound loaded on the material.

### Example 5. A High Purity Polygonum cuspidatum Extract (>95% Trans-Reveratrol)

Trans-resveratrol is extracted from the root of Polygonum cuspidatum (Reynoutria japonica). The trans-resveratrol powder is a crystalline material as shown by the X-ray reflection in FIG. 8.

Characterization of the amorphous or crystalline state of trans-resveratrol raw material was performed by X-ray diffraction (XRD) using a Bruker D2 Phaser diffractometer with Cu-Kα radiation. Samples were run over the range of 6° to 50° (2θ) with a step size of 0.04° at a speed of 2°/min.

### Example 6. Solid Dispersion of Trans-Resveratrol and the Bamboo Silica Extract

The bamboo silica extract, as described in example 1, and the trans-resveratrol, as described in example 5, are ball milled, in a ratio by weight of 70/30 to obtain a trans-resveratrol based solid dispersion according to the process conditions described in example 3.

The trans-resveratrol based solid dispersion with the bamboo silica extract as carrier (also referred to herein as composition 303DS06) has been characterized by X-ray diffraction and the reflections obtained have been compared to a physical blend of the bamboo silica extract and trans-resveratrol pure crystalline material.

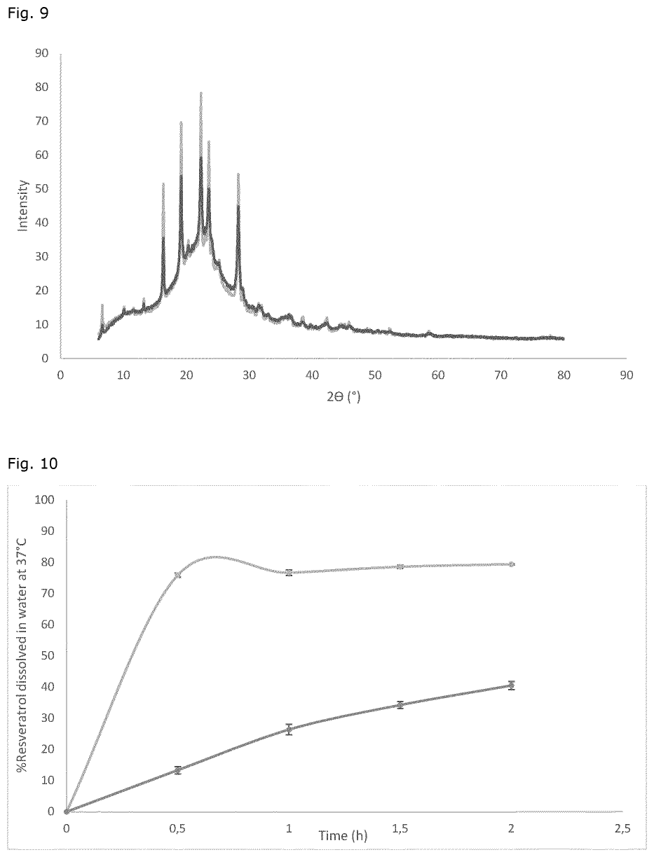

The comparison of the X-ray reflections of 303DS06 and of the physical blend among trans-resveratrol pure crystalline material and bamboo silica extract shows that the ball-milling process amorphized the trans-resveratrol as the intensities of the characteristics trans-resveratrol X-ray reflections decrease after the ball milling process (FIG. 9).

### Example 7. Comparison of the Trans-Resveratrol Kinetic Release

The trans-resveratrol kinetic release of trans-resveratrol solid dispersion obtained by ball-milling with bamboo silica extract as carrier (example 6) has been compared to the trans-resveratrol kinetic release of the trans-resveratrol pure crystalline material (example 5) in the conditions of release described in example 4, with a dose of 50 mg trans-resveratrol per sample.

The comparison of the tans-resveratrol kinetics release of both products is shown in FIG. 10. The release trial shows that the amount of trans-resveratrol dissolved for the pure crystalline material reaches 40 w % of active dissolved after 2 hours at 37° C. in water. For the trans-resveratrol solid dispersion with the bamboo silica extract as carrier (303DS06) this amount reaches approximately 80 w %. Hence, the amount of trans-resveratrol dissolved after 2 hours in water at 37° C. is increased by a factor 2 when it is in the form of a solid dispersion with the bamboo silica extract as carrier in comparison to the pure crystalline material.

Next, we compared the release kinetics of trans-resveratrol of the solid dispersion with the bamboo silica extract as carrier (303DS06) to the solid dispersion of trans-resveratrol with a synthetic amorphous mesoporous silica OSI100 (also referred to herein as composition 303DS05) with high specific surface area (680 m²/g). The same trans-resveratrol content (30 w %) was used and the comparison was performed in the conditions of release described in example 4.

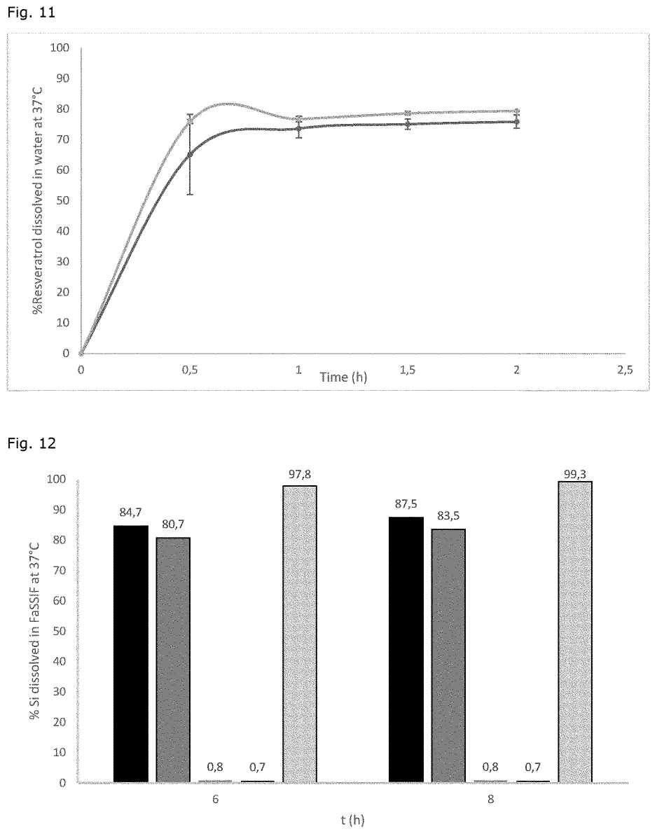

The comparison of the trans-resveratrol kinetics release of both products is shown in FIG. 11.

The kinetics release of both trans-resveratrol based solid dispersion materials obtained with a synthetic amorphous mesoporous silica (OSI100) with a high specific surface area of 680 m²/g and the one obtained with the bamboo silica extract are comparable. The profiles of the kinetic curves are similar, and the amount released after 2 hours is between 75 to 80% in both cases considering the standard deviation of the trials (FIG. 11).

These data confirms the surprising results from example 4. In general a higher specific surface area of a mesoporous material is directly correlated to a higher release of a compound loaded on the material. Hence, composition 303DS05 comprising the synthetic amorphous mesoporous silica (OSI100) with a surface area of 680 m²/g would be expected to release significantly more of the loaded compound compared to composition 303DS06 comprising the bamboo mesoporous silica with a surface area of around 350-400 m²/g. Yet, the compositions comprising the bamboo mesoporous silica released comparable amounts of the loaded compound compared to the synthetic silica containing compositions, with an even higher release during the first hour, for both compounds tested.

### Example 8. Bamboo Silica Extract as Source of Bioavailable Silicon

Next, we investigated whether the bamboo silica extract could also be used as source for bioavailable silicon. It is known that silica is not easily absorbed by the body. In order to make silicon more bioavailable, it needs to be broken down in smaller, more soluble forms. Calcium and magnesium are both known to help break down silica into more bioavailable forms of silicon. This is because calcium and magnesium ions have a higher affinity for oxygen than silicon does, so they can help to displace the oxygen atoms in silica and release the silicon.

Therefore blends were made with Aquamin TG, a natural and highly bioactive source of minerals rich in calcium and magnesium. Aquamin TG is produced from calcareous marine algae (Lithothamnion sp.) harvested from the North Atlantic sea-bed.

The composition of Aquamin TG is subject to seasonal variations. However, some typical characteristics and values are listed below.

TABLE 2
summarizes the main physical and chemical
properties of the Aquamin TG algae extract.

| Property | Value | |
|---|---|---|
| Calcium content | 30% | min |
| Magnesium content | 2.2% | min |
| Moisture | 5% | max |
| Particle size | 500 | μm (max) |
| Bulk density | 0.7-0.9 | g/cm³ |

Several compositions were evaluated, wherein unmodified maize starch was added as a granulating agent.

Composition A:

| Ingredients | Description | ratio (w %) |
|---|---|---|
| Bamboo extract with | Mesoporous silica (>75%) | 55 |
| a content of min. 75% | Origin Synthesis: Bambusa arundinaceae; | |
| mesoporous silica. | Plant part used: young shoots | |
| Aquamin TG | Aquamin TG is produced from a natural mineral | 36 |
| LITHOS INGREDIENT | source - calcareous marine algae (Lithothamnion | |
| | sp.) | |
| CompactCel ® | Separating agent which loosens up powder blends | 9 |
| F300.23 FLO | and improves the flowability. | |

Composition B:

| Ingredients | Description | ratio (w %) |
|---|---|---|
| Bamboo extract with | Mesoporous silica (>75%) | 29 |
| a content of min. 75% | Origin Synthesis: Bambusa arundinaceae; | |
| mesoporous silica. | Plant part used: young shoots | |
| Aquamin TG | Aquamin TG is produced from a natural mineral | 66 |
| LITHOS INGREDIENT | source - calcareous marine algae (Lithothamnion | |
| | sp.) | |
| CompactCel ® | Separating agent which loosens up powder blends | 5 |
| F300.23 FLO | and improves the flowability. | |

Composition C:

| Ingredients | Description | ratio (w %) |
|---|---|---|
| Bamboo extract with a | Mesoporous silica (>75%) | 100 |
| content of min. 75% | Origin Synthesis: Bambusa arundinaceae; | |
| mesoporous silica. | Plant part used: young shoots | |

Composition D:

| Ingredients | Description | ratio (w %) |
|---|---|---|
| Bamboo extract with | Silica (>75%) | 58 |
| a content of min. 75% | Origin Synthesis: Bambusa arundinacea | |
| silica. | Exudate extract | |
| | Plant part used: Dried resin (=exudate cleared by | |
| | boiling) | |
| Aquamin TG | Aquamin TG is produced from a natural mineral | 40 |
| LITHOS INGREDIENT | source - calcareous marine algae (Lithothamnion | |
| | sp.) | |
| CompactCel ® | Separating agent which loosens up powder blends | 2 |
| F300.23 FLO | and improves the flowability. | |

The degradation behavior of the different compositions were characterized by ICP-OES (5110 VDV from Agilent) to assess the amount of degraded silica in function of time, using the following conditions:
Apparatus: European Pharmacopoeia dissolution apparatus type II (paddles)—AT Xtend (Sotax)

Dissolution medium: FaSSIF (Fasted State Simulated Intestinal Fluid)

Volume medium: 900 ml

Speed of rotation: 75 rpm
Stirrer: paddles
Temperature medium: 37.0° C.±0.5° C.
Sampling volume: 5 ml filtered on PTFE membrane (0.45 μm)-syringe filter. Fresh medium (FaSSIF) is added on each flask where the sampling has been performed.
Sampling frequency: 6 h and 8 h

No specific sample preparation has been performed after the sampling during the dissolution trial. Calibration curves in FaSSIF medium have been prepared with Si (silicon) concentration ranging from 1.5 to 25 ppm.

The results are expressed in percentage of the amount of silicon dissolved and calculated in function of the amount of silicon introduced in the medium.

The percentage of Si dissolved for the compositions A, B, C and D, have been evaluated by dissolution trials and the results are presented in FIG. 12.

The bamboo silica extract derived from young shoots and comprising >70% mesoporous silica (Composition C) did not dissolve after 8 hours in aqueous media (FaSSIF). Less than 1% silicon was released (FIG. 12). However, when the bamboo silica extract was blended with Aquamin TG as in composition A (36% Aquamin TG), the silicon amount dissolved reached approximately 88 w % after 8 hours in FaSSIF at 37° C. Interestingly, composition D comprising the bamboo extract from exudate and Aquamin TG in the same silica/Aquamin in the ratio as in composition A, did hardly release any silicon in the dissolution trial (FIG. 12).

The only parameter which is differencing composition A and D is the origin of the bamboo silica extract. The one used in the composition A comes from young bamboo shoots. The silica present is mesoporous silica. The one used in the composition D comes from exudates and the silica present is non-mesoporous.

The origin of the bamboo silica extract has a high influence on the dissolution of these silicas in the materials in presence of alkali or alkaline-earth metals (Ca and Mg).

Additionally, when the amount of Aquamin TG increased as in composition B (66 w % Aquamin TG) compared to composition A (36 w % Aquamin TG), the silicon amount dissolved decreased from 87.5 w % to 83.5 w % after 8 hours in FaSSIF at 37° C. These results indicate that the amount of dissolved silicium is related to the amount of lithothamnion introduced into the medium and that the expected optimum amount of dissolved silicium should be reached when the amount of lithothamnion is between and 55% of the mixture. For higher amounts of lithothamnion (50-90%), the amount of dissolved silicium decreases.

### Example 9. Milling of Bamboo Extract Further Increases the Bioavailability of Silicon

In order to obtain a product with all-natural product where the silica content is completely soluble, the bamboo silica extract from young shoots has been processed by ball-milling to decrease the particle size. By decreasing the particle size, the contact surface area is increased and the silicium dissolution kinetic should be enhanced.

The bamboo silica extracted obtained after ball-milling process in the conditions described previously in Example 3, has been used in combination with Aquamin TG describing the composition E.

Composition E:

| Ingredients | Description | ratio (w %) |
|---|---|---|
| Milled bamboo extract | Mesoporous silica (>75%) | 55 |
| with a content of min. | Origin Synthesis: Bambusa arundinaceae; | |
| 75% silica. | Plant part used: young shoots | |
| Aquamin TG | Aquamin TG is produced from a natural mineral | 36 |
| LITHOS INGREDIENT | source - calcareous marine algae (Lithothamnion | |
| | sp.) | |
| CompactCel ® F300.23 | Separating agent which loosens up powder blends | 9 |
| FLO | and improves the flowability. | |

The particle size distribution of the bamboo silica extract from young shoots after ball-milling has been determined by a particle size analyzer with the Beckman Coulter method in water at room temperature. The particle size distribution in volume of the material is centered at 14.50 μm. More specifically, the following parameters of the particle size distribution of this material were obtained:

TABLE 3
Main parameters of the particle size distribution in volume of
the bamboo silica extract from young shoots after ball-milling.

| d₁₀ (μm) | d₂₅ (μm) | d₅₀ (μm) | d₇₅ (μm) | d₉₀ (μm) |
|---|---|---|---|---|
| 3.34 | 6.67 | 14.49 | 31.39 | 79.44 |

After ball-milling, the median particle size of the bamboo silica extract is decreased by a factor 7.4 when comparing the d50 before (107 μm) and after ball-milling (14.49 μm). Subsequently, the percentage of Si dissolved for composition E was evaluated by the dissolution trial.

As shown in FIG. 12, reducing the particle size of the bamboo silica extract by ball-milling further increased the percentage of silicium dissolved, more particularly by approximately 13 w % after 8 hours in FaSSIF compared to composition A. Interestingly, in the conditions of Composition E, the silicium content is completely dissolved after 8 hours in FaSSIF at 37° C.

### Example 10. Dissolution of Silicon from a Bamboo Silica Extract at Different Temperatures and in the Presence of a Calcium Salt

Finally, the dissolution kinetics of the bamboo silica extract from young shouts was studied at different temperatures. For this purpose a composition F was made of which the specifics are detailed below.

945 mg of composition F was introduced in 500 ml of ultra-pure water at the following temperatures: 23° C.; 41° C.; 50° C. and 60° C.

After 0.5 h; 1 h; 2 h and 3 h a sampling of 5 ml was performed and the product was filtered on polytetrafluoroethylene (PTFE) filter (0.45 μm).

Composition F:

| Ingredients | Description | ratio (w %) |
|---|---|---|
| Bamboo extract with | Mesoporous silica (>75%) | 38 |
| a content of min. 75% | Origin Synthesis: Bambusa arundinaceae; | |
| silica. | Plant part used: young shoots | |
| Calcium acetate | CAS: 62-54-4 | 62 |
| | E263 | |
| | C₄H₆CaO₄ | |

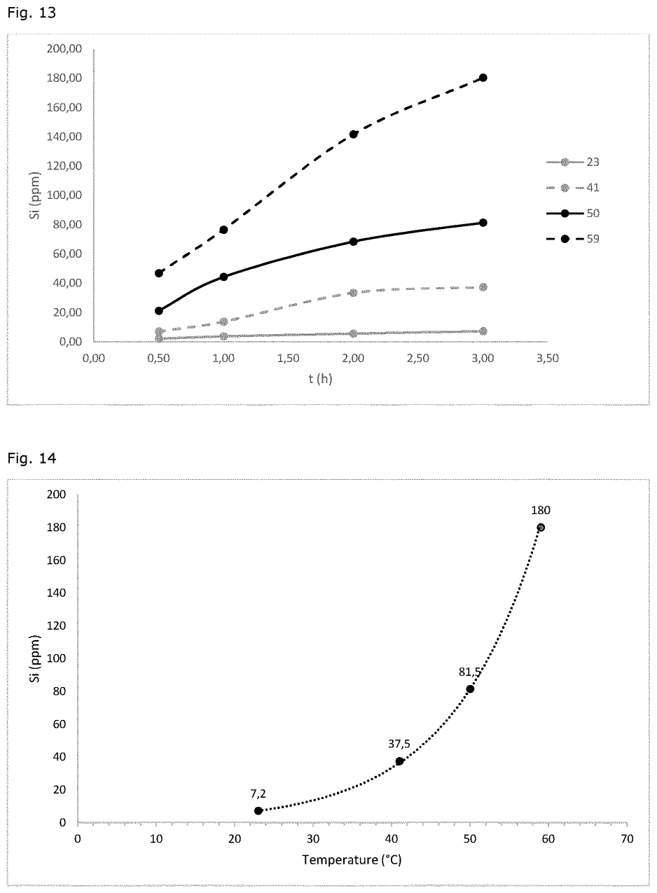

The amount of dissolved silicium in the samples was evaluated by ICP-OES with a calibration curve where the silicium concentration ranged from 0 to 50 ppm Si. The samples were diluted 10 times in ultrapure water before analysis. The kinetics dissolution curves for each temperature studied are shown in FIG. 13.

The evolution of the Si content in ultrapure water of composition F is highlighted in FIG. 14 and follows an exponential trend in function of the temperature.

### Example 11—Influence of Milling Conditions on the Solubility and Resulting Composition

A commercially available silica extract from bamboo was analyzed, and consequently ball-milled at varying conditions. The silica extract contained 68% silica (SiO₂).

100 mg of the bamboo extract was ball milled at a BPR ratio of 7.6 at a fixed RPM of 450 for 4 hours. The starting and resulting compositions were analysed and placed in FaSSIF at 37° C. for 4, 6 and 8 hours. The amount of dissolved silicium was measured by ICP-OES. The dissolved amount of silicium is elemental silicium (Si), not silica (SiO₂).

The silica extract prior to milling had a specific surface area of 288 m²/g. After 2 hours of ball-milling, the specific surface area was reduced to 172 m²/g indicating the destructive effect of ball milling on highly porous structures. All calculated according to BET-theory.

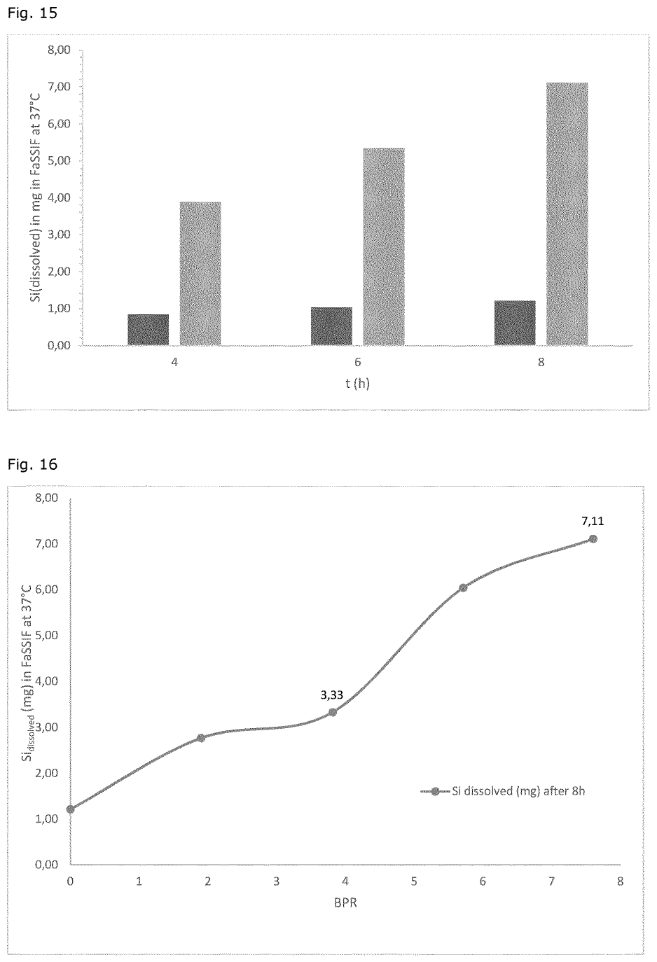

FIG. 15 displays the amount of silicium (mg) dissolved in FaSSIF at 37° C. after 4, 6 and 8 hours, before and after the extracts were ball milled for 8 hours (7.6 BPR, 450 RPM, 8 hours milling time). It is clear that ball milling significantly increased the amount of silicium dissolved in FaSSIF in each of these timeframes.

The experiment was repeated at a fixed milling time of 4 hours, with varying BPR ratio, at a fixed RPM of 450. The resulting composition was analysed and placed in FaSSIF at 37° C. for 8 hours. The amount of dissolved silicium was investigated.

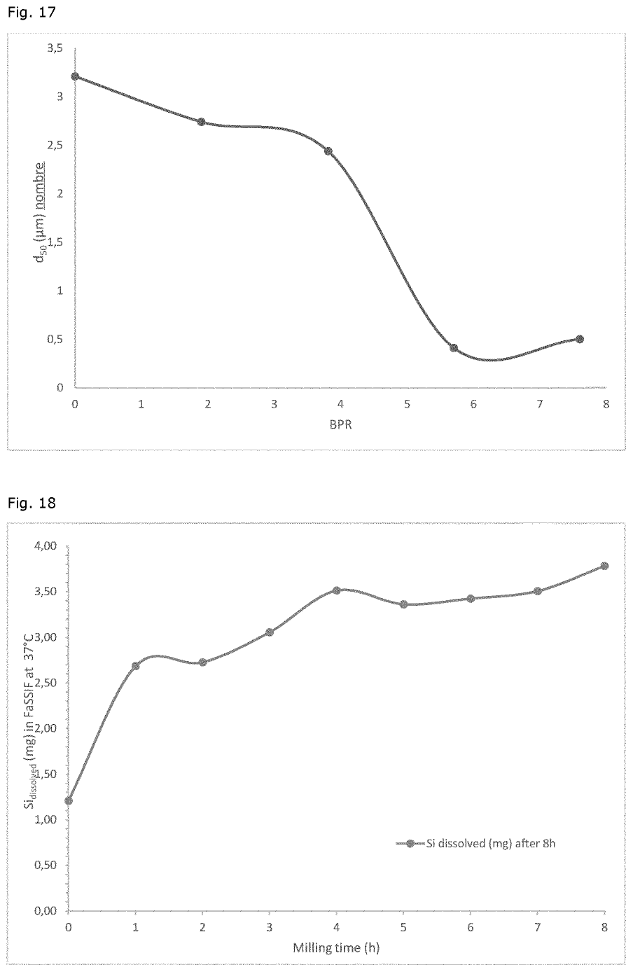

FIG. 16 displays the amount of silicium (mg) dissolved in FaSSIF at 37° C. after 8 hours, in function of the BPR ratio at the aforementioned milling conditions (450 RPM, 4 hours milling time). FIG. 17 displays the median particle size in function of the BPR ratio at the same milling conditions. An optimum is obtained at a BPR ratio of 5.5. At that point, further increases to the dissolution rate and/or particle size are limited especially in view of the rapidly increasing energy requirements at such high BPR ratios.

The experiment was repeated at a BPR ratio of 3.8, with a fixed 450 RPM. The milling duration was varied. FIG. 18 displays the amount of silicium (mg) dissolved in FaSSiF at 37° C. after 8 hours, in function of the milling time (h). For this BPR ratio and RPM, it is clear that an optimum in milling time is obtained around 4 hours. Further increases to milling time do not result in benefits to the silicium dissolution rate.

### Example 12—Addition of Calcium Salts

The influence of calcium salts on a range of mesoporous silicas with varying specific surface areas was investigated. When the specific surface area of said silica was above 650 m²/g, a very significant increase was observed. In fact, very high dissolution rates of silicium could be obtained. However, when the specific surface area of silica was below 550 m²/g, the increase upon addition of calcium was limited. For silica with a surface area below 350 m²/g, almost no benefit from the addition of calcium was observed. Without being bound by theory, it is believed dissolved Ca²⁺ ions affect the dissolution and gelling kinetics at the water-silica interface and particularly destabilize structures with a high specific surface area.

1. A composition comprising a silica extract derived from bamboo, wherein said composition is characterized by a dissolution of silicium (Si) of at least 3 wt. % relative to the silicium in the composition, in FaSSIF after 8 hours at 37° C. as measured by ICP-OES.

2. The composition according to claim 1, wherein said composition is characterized by a dissolution of silicium (Si) of at least 5 wt. % relative to the silicium in the composition, in FaSSIF after 8 hours at 37° C. as measured by ICP-OES.

3. The composition according to claim 1, wherein said composition is characterized by a dissolution of silicium (Si) of at least 7 wt. % relative to the silicium in the composition, in FaSSIF after 8 hours at 37° C. as measured by ICP-OES.

4. The composition according to claim 1, wherein said composition comprises silica SiO2 in an amount of at least 50 wt. %.

5. The composition according to claim 1, wherein said composition has a median particle size d50 between 0.05 and 1.00 μm as determined by Beckman Coulter method in water at room temperature.

6. The composition according to claim 5, wherein the median particle size d50 is between 0.1 and 0.5 μm.

7. The composition according to claim 1, wherein said composition has a specific surface area calculated from nitrogen sorption isotherms at liquid nitrogen temperature (77K) by the Brunauer, Emmett and Teller (BET) theory, of at most 600 m²/g.

8. A method for the production of a soluble silica composition, the method comprising:
providing a milling composition, said composition comprising a silica extract derived from bamboo in an amount of at least 30 wt. % relative to the composition; and
ball milling said milling composition, thereby obtaining a soluble silica composition.

9. The method according to claim 8, wherein ball milling is performed at a ball to powder (BPR) ratio by weight between 3.0 and 8.0.

10. The method according to claim 8, wherein the milling time is between 3.0 and 8.0 hours.

11. The method according to claim 8, wherein the median particle size d50 of the soluble silica composition is between 0.1 and 0.5 μm as determined by Beckman Coulter method in water at room temperature.

12. The method according to claim 8, wherein the median particle size d50 of the milling composition is higher than 2.5 μm as determined by Beckman Coulter method in water at room temperature.

13. The method according to claim 8, wherein the soluble silica composition has a specific surface area calculated from nitrogen sorption isotherms at liquid nitrogen temperature (77K) by the Brunauer, Emmett and Teller (BET) theory between 100 m²/g and 300 m²/g.

14. The method according to claim 8, wherein the milling composition has a specific surface area calculated from nitrogen sorption isotherms at liquid nitrogen temperature (77K) by the Brunauer, Emmett and Teller (BET) theory above 250 m²/g.

15. A method for the production of a soluble silica composition, the method comprising the steps of:
providing an insoluble composition, said composition comprising a silica extract derived from bamboo in an amount of at least 30 wt. % relative to the composition; and
measuring the specific surface area calculated from nitrogen sorption isotherms at liquid nitrogen temperature (77K) by the Brunauer, Emmett and Teller (BET) theory of said insoluble composition;
when the specific surface area of the insoluble composition is above 650 m²/g, a calcium salt is added to the insoluble composition in an amount of at least 10 wt. % relative to the weight of the silica extract; thereby obtaining the soluble silica composition; and
when the specific surface area of the insoluble composition is below 650 m²/g, said insoluble composition is ball milled thereby obtaining the soluble silica composition.

16. The composition according to claim 7, wherein the specific surface area is between 100 m²/g and 300 m²/g.

17. The method according to claim 9, wherein the ball to powder (BPR) ratio by weight is between 3.5 and 7.0.

18. The method according to claim 10, wherein the milling time is between 3.5 and 5.0 hours.
